(19) United States
(12) Patent Application Publication
(10) Pub. No.: US 2022/0280411 A1
(43) Pub. Date: Sep. 8, 2022

(54) Mattifying Cosmetic Compositions

(71) Applicant: ELC Management LLC, Melville, NY (US)

(72) Inventor: Wilson A. Lee, Hauppauge, NY (US)

(21) Appl. No.: 17/189,768

(22) Filed: Mar. 2, 2021

(51) Int. Cl. A61K 8/81; A61K 8/25; A61K 8/34; A61Q 19/00; A61K 8/02; A61K 8/87

(52) U.S. Cl. CPC A61K 8/8152; A61K 8/25; A61K 8/345; A61Q 19/00; A61K 8/02; A61K 8/87; A61K 2800/43; A61K 2800/48

(57) ABSTRACT

Mattifying single phase cosmetic compositions that are flexible and resistant to water below a selected temperature, 43° C. for example. The compositions wear well, are smudge and flake resistant, as well as oil resistant. Compositions according to the invention comprise specific combinations of acrylates/VA copolymer, acrylates copolymer and HDI/trimethylol hexyllactone crosspolymer/silica in a cosmetically acceptable base or delivery vehicle. Compositions according to the invention are hydrophilic before application, but dry to a clear hydrophobic film. The dried film can be easily removed when scrubbed with water above a certain temperature, but not as easily removed with water below that temperature. The compositions are easy to manufacture, flexible and comfortable, and suitable for use on skin and hair.

### FIELD OF THE INVENTION

The present invention is in the field of personal care products and cosmetics for keratinic surfaces, such as the skin. Specifically, the invention pertains to lotions, serums and creams that provide a mattifying or blurring effect on the skin.

### BACKGROUND

Personal care and cosmetic products come in a variety of forms, which vary depending upon the look desired by the user. Specifically, products may be matte or glossy. The look conferred by a high gloss (high shine) cosmetic product is considered glamorous and sensual. However, high shine products have tended to have little staying power on the skin. Flaking and smudging are common problems with high shine products, as well discomfort due to a lack flexibility of the product on the skin. These issues were successfully addressed in co-owned, co-pending applications U.S. Ser. No. 15/632,903 and U.S. Ser. No. 17/176,527, herein incorporated by reference in their entirety. Disclosed therein, are high shine color cosmetic compositions that are flexible and resistant to water below 43° C. The compositions wear well, are smudge and flake resistant, as well as oil resistant, making them very suitable as high shine, long wear cosmetics. Those compositions comprise specific combinations of acrylates/VA copolymer and acrylates copolymer in a cosmetically acceptable base or delivery vehicle. The compositions are hydrophilic and easily removed when scrubbed with water above a certain temperature, but not as easily removed with water below that temperature. The compositions are easy to manufacture, flexible and comfortable, and suitable for use on keratinic surfaces, such as the skin, hair and nails. However, the compositions described in those applications were inherently glossy.

At the present time, a need has developed for mattifying compositions that have many of the same properties as the glossy compositions disclosed in U.S. Ser. No. 15/632,903 and U.S. Ser. No. 17/176,527. It will be especially advantageous to provide mattifying cosmetic compositions that offer a blurring, or soft focus effect and true color, as well as easy removal with warm water. The present invention provides such compositions.

Cosmetic compositions that comprise acrylates polymers have typically been provided in the form of emulsions containing oils, surfactants, and/or emulsifiers or anhydrous formulations containing volatile components such as oils or alcohols. For example, U.S. Pat. No. 7,323,162 discloses silicone in water emulsions that comprises a water phase, an oil phase, and two kinds of film formers (a water-soluble, oil resistant film former, such as Covacryl A15 or E14, and an oil soluble (water resistant) film former, at least one of which is a particular silicone-modified acrylates copolymer. The compositions further include a surfactant which is specifically adapted to stabilize silicone in water emulsions. In contrast, co-owned U.S. Pat. No. 8,932,570 discloses transfer-resistant, single phase aqueous cosmetic compositions. The compositions consist essentially of 1%-95% of a water-soluble, film-forming acrylates copolymer and 1%-60% of a water-soluble plasticizer for the copolymer, and, optionally, 1%-20% pigment. The acrylates copolymer, consists essentially of a monomer selected from the group consisting of acrylic acid and methacrylic acid and a co-monomer selected from the group consisting of alkyl and alkoxyl acrylates and alkyl and alkoxyl methacrylates. The plasticizer may be selected from polyether derivatives, polyoxypropylene derivatives, glycol and glycol derivatives and glycerin and glycerin derivatives, and combinations thereof. The compositions contain no oils, waxes, surfactants or emulsifiers, are water- and oil-resistant upon drying on the skin, and exhibit high gloss and long-wear and transfer-resistant properties. When these compositions contain pigments, they also demonstrate high color intensity. In contrast with two phase oil and water emulsion systems, these compositions are less complex and less costly to formulate, requiring only a single aqueous phase, and no oils, surfactants, or emulsifiers. Additionally, these compositions may be formulated with a single type of water-soluble film former.

To the best of our knowledge, the prior art fails to disclose a mattifying composition comprising 8% to 16% of acrylates/VA copolymer, 0.2% to 0.8% of acrylates copolymer, and 1% to 15% of hydrophobic powder, such as HDI/trimethylol hexyllactone crosspolymer/silica, as disclosed herein. Nor does it disclose ratios of acrylates/VA copolymer and acrylates copolymer, as disclosed herein, nor their usefulness.

### SUMMARY

The present invention provides a mattifying cosmetic composition, with or without color, that is flexible and resistant to water below 43° C. The compositions provide a blurring effect that diminishes the appearance of fine lines in the skin. Compositions according to the invention comprise specific combinations of acrylates/VA copolymer, acrylates copolymer and hydrophobic powder (particularly HDI/trimethylol hexyllactone crosspolymer/silica) in a cosmetically acceptable base or delivery vehicle. Compositions according to the invention are single phase, hydrophilic before and during use, but hydrophobic upon drying. Nevertheless, the dry film is easily removed when scrubbed with water above a certain temperature, but not as easily removed with water below that temperature. The compositions are easy to manufacture, flexible and comfortable, and suitable for use on skin.

### DETAILED DESCRIPTION

Except in operating and comparative examples, or where otherwise explicitly indicated, all numbers in this description indicating amounts or ratios of material or conditions of reaction, physical properties of materials and/or use are to be understood as modified by the word “about.” All amounts are presented as percentages by weight of the final composition, unless otherwise specified.

Throughout the present specification, “film former” or the like refers to a polymer leaves a film on the substrate to which it is applied, for example, after a solvent accompanying the film former has evaporated, absorbed into and/or dissipated on the substrate.

“True color” compositions are those in which the color of the applied composition, after a period of time, remains the same or substantially the same as at the time of application to the skin or hair.

A “flexible” composition is one that when applied to the skin or hair for its intended use, does not crack or flake for a defined period of time, such as four hours or eight hours of wear. If a composition is not adequately flexible, then it is “rigid”.

“Water resistant” means that a composition deposited on the skin or hair, after it has been allowed to dry or cure, does not dissolve or re-wet or absorb moisture or be otherwise adversely affected by the water.

“Single phase” implies that the composition is not a water-in-oil or oil-in-water emulsion.

“Comprising” and the like, mean that a list of elements may not be limited to those explicitly recited.

### Acrylates/VA Copolymer

A first main ingredient of the invention is acrylates/VA copolymer (INCI name), C₁₅H₂₆O₄, also known as ethenyl acetate or 2-ethylhexyl prop-2-enoate (IUPAC names); CAS number 25067-02-1. For detailed information, see PubChem Compound Database; CID=168269.

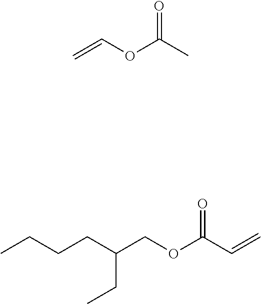

In cosmetics, this material often functions as a binder, film former, adhesive and/or hair fixative. When deployed in aqueous cosmetic systems acrylates/VA copolymer can impart a film on the skin or hair. The pure acrylates/VA copolymer film features a temperature dependence, such that a water rinse of about 40° C. or more will degrade the film, and allow it to be removed from a surface, while retaining its integrity at temperatures at or below normal skin temperature (i.e. 36.5-37.5° C.).

Compositions of the invention typically comprise 8% to 16% of acrylates/VA copolymer by total weight of the composition, for example 11% to 13% by total weight of the composition. Acrylates/VA copolymer is commercially available as an aqueous mixture. For example, Daido Chemical Corp. offers Vinysol 2140L is a 46.6% aqueous mixture of acrylates/VA copolymer, and Vinysol 2140LP is a 47.5% aqueous mixture of acrylates/VA copolymer.

The strength of acrylates/VA copolymer films makes this material suitable for thinly applied cosmetics that will not crack or flake easily. However, without modification, acrylates/VA copolymer tends to make the final product too rigid to be commercially useful.

### Acrylates Copolymer

To address the problem of high rigidity, the acrylates/VA copolymer was combined with an acrylates polymer that has a lower T_{g} than acrylates/VA copolymer. In general, a lower T_{g} provides more flexibility to the resulting film. In the present invention, to provide a suitable amount of flexibility in the dried composition, a second main ingredient is acrylates copolymer, C₁₄H₂₂O₆, also known as ethyl prop-2-enoate; methyl 2-methylprop-2-enoate or 2-methylprop-2-enoic acid (IUPAC names); CAS number 25133-97-5. For detailed information, see PubChem Compound Database; CID=168299. In various types of cosmetic formulations, acrylates copolymer has a wide variety of uses including as film formers, hair fixatives, binders, and suspending agents, viscosity enhancers, antistatic agents and adhesives.

As noted above, acrylates/VA copolymer films feature a temperature dependence, such that a water rinse of at least about 40° C. will degrade the film, but not below this. In combining acrylates/VA copolymer with acrylates copolymer at the ratios disclosed herein, it was noted that the resulting films exhibit a different minimum temperature of water that is required to degrade the film. Specifically, the addition of acrylates copolymer tends to increase the minimum temperature of water that is required to degrade the film. In the United States, typical water temperatures from a home faucet are set not to exceed 120° F. (48.9° C.). Therefore, the certain minimum temperature should be set between 40° C. and 48.9° C., preferably from 42° C. to 46° C., more preferably from 43° C. to 44° C. In various embodiments of the present invention, the certain minimum temperature has been adjusted to be from 43° C. to 44° C. 43° C. to 44° is most preferred because it is several degrees warmer than normal, healthy skin temperature (i.e. 36.5-37.5° C.), but not so high as to damage the skin or cause pain. The minimum temperature of 40° C. reported for the Vinysol 2140L material, is close to normal skin temperature and may not provide a consistent experience in consumer use. By using acrylates/VA copolymer in combination with acrylates copolymer (and with a hydrophobic powder, see below), in the ratios disclosed herein, the minimum temperature can be fixed to more than 40° C., preferably 42° to 46° C. to provide more of a margin of error, or better 43° C. to 44° C.

In the present invention, useful concentrations of acrylates copolymer are from 0.2% to 0.8% based on total weight of the composition; for example 0.3% to 0.7%, for example 0.4%, 0.6%, for example 0.5%. In co-pending applications U.S. Ser. No. 15/632,903 and U.S. Ser. No. 17/176,527 it was reported that at concentrations of acrylates copolymer below about 0.5%, the acrylates copolymer cannot impart enough flexibility to the final composition. However, we have discovered that the addition of hydrophobic powder to the composition lowers the required concentration of acrylates copolymer, because hydrophobic powder contributes some plasticizing effect on the acrylates/VA copolymer (as well as other properties to be described below).

Based on the above, we can say that the ratio of the weight of acrylates/VA copolymer to the weight of acrylates copolymer must be in the range 10:1 to 80:1, preferably 10:1 to 60:1, more preferably 12:1 to 40:1. Furthermore, all weight ratios of acrylates/VA copolymer to acrylates copolymer in between 10:1 and 80:1 are useful, and encompassed by the present invention. These useful ratios include, for example, X:1, where X is any integer or half-integer from 10 to 80. For example, X may be any integer or half-integer from between 10 and 60, or any integer or half-integer between 12 and 40.

Acrylates copolymer is commercially available as an aqueous mixture. For example, Daito Kasei Kogyo Co. offers Daitosol 5000AD and Interpolymer Corporation offers Syntran 5710. Daitosol 5000AD is a 50% aqueous mixture of acrylates copolymer. Syntran 5710 is a 40.8% aqueous mixture of acrylates copolymer.

### Hydrophobic Powder

The formulation system of the present invention is particularly good at dispersing hydrophobic powders in an aqueous system. Examples include silica and titanium. Of particular interest, however, is HDI/trimethylol hexyllactone crosspolymer/silica (INCI name). This material is an oil soluble microsphere powder used for its silky texture and enhanced slip, as well as for its light diffusing or mattifying effects, which reduce the appearance of fine lines in the skin. Being hydrophobic, HDI/trimethylol hexyllactone crosspolymer/silica generally requires a volatile solvent; silicone and alcohol being just two examples. However, the use of hydrophobic powders and volatile solvents introduces well known problems, such as clogging the pores of the skin, as well as producing an ashy appearance and unpleasant drying effect.

It has now been discovered that the mattifying benefits of HDI/trimethylol hexyllactone crosspolymer/silica can be obtained while avoiding the unpleasant side effects mentioned above. This is achieved by using HDI/trimethylol hexyllactone crosspolymer/silica in conjunction with acrylates copolymer and acrylates/VA copolymer, as described herein. No volatile solvent is needed. It was not at all clear that the combination acrylates copolymer and acrylates/VA copolymer, as described herein, could secure the mattifying benefits of HDI/trimethylol hexyllactone crosspolymer/silica powder without the use of a volatile solvent. There was nothing in the literature on HDI/trimethylol hexyllactone crosspolymer/silica powder to suggest as much. Nevertheless, when used as disclosed herein, the HDI/trimethylol hexyllactone crosspolymer/silica powder is not able to clog the pores of the skin, does not have an ashy appearance or drying feel, but does provide a desirable mattifying or blurring effect. Furthermore, as demonstrated in co-pending applications U.S. Ser. No. 15/632,903 and U.S. Ser. No. 17/176,527, one advantageous feature of compositions of the present invention is that they are hydrophilic before and during use, but hydrophobic upon drying. It was not clear that the addition of HDI/trimethylol hexyllactone crosspolymer/silica powder would not destroy this and other unique properties of the combination acrylates copolymer and acrylates/VA copolymer. Surprisingly, we have discovered how to retain those unique properties by adjusting the concentrations and ratios of acrylates/VA copolymer and acrylates copolymer.

In the present invention, useful concentrations of hydrophobic powders, such as HDI/trimethylol hexyllactone crosspolymer/silica, are from 1% to 15%, preferably from 4% to 8%, more preferably from 3% to 6%. HDI/trimethylol hexyllactone crosspolymer/silica is commercially available. BPD-500 from Kobo Products, Inc. and D-800 from Nikko Chemicals are two examples.

### Water

Compositions have a single aqueous phase, and comprise 40% to 70% of water, preferably 50% to 60% of water, by weight of the total composition, and have no oil or silicone. The amount of water is that from all sources, such as in commercially available aqueous dispersions of acrylates/VA copolymer and acrylates copolymer. The ability to formulate in an aqueous, hydrophilic state that dries to a hydrophobic state (while having other beneficial properties described herein) is a great advantage of the present invention. While the composition is in a first or hydrophilic state, the ability to formulate with water soluble ingredients is enhanced, and application of the cosmetic is easier and feels nicer. When dried to a second or hydrophobic state, the applied composition resists breakdown from moisture in the skin and atmosphere. However, unlike anything disclosed in U.S. Pat. No. 8,932,570, the dried compositions of the present invention may be easily washed off with water at or above that certain minimum temperature and an application of shear. Both shear and a certain minimum water temperature are needed to remove the composition from the skin or hair. For example, when the dried composition is exposed to water at or above a certain minimum temperature, the composition experiences a breakdown in structure, but does not otherwise dissolve in the applied water, so that the composition remains on the skin or hair. Likewise, when the dried composition is exposed to shear (in the form of a typical vigorous scrubbing action), without water or with water below a certain minimum temperature, the composition remains in place, having excellent adhesion to the skin or hair. To effect the removal of the composition from the skin or hair, both shear (in the form of a typical vigorous scrubbing action) and water above a certain minimum temperature must be applied to the composition in order to lift it off of the skin.

### Forms

Compositions of the invention may be implemented as lotions, serums and creams. The viscosity of the final composition can be adjusted with thickeners, but may be sufficiently low to permit the composition to be dispensed as a spray, using a cosmetic or personal care-type dispensing system, such as a manual spay pump or pressurized container.

### Other Ingredients

Various ingredients are optional, but may be included in the cosmetic compositions of the invention to fine tune the consumer experience or enhance the performance of the composition. At the levels disclosed herein, the following ingredients do not seem to adversely affect the cosmetic and commercial properties of the compositions.

When the composition is intended to impart color, the composition may comprise from 0% to 20% of hydrophilic pigments. Compositions of the invention with pigments offer “true color,” that is the color of the composition upon drying remains the same or substantially the same as at the time of wet application to the skin or hair.

Optionally, suspending agents typically considered safe in cosmetic products, such as xanthan gum, carrageenan and microcellulose, may be useful to ensure that the HDI/trimethylol hexyllactone crosspolymer/silica, or other hydrophobic powder, remains evenly dispersed throughout the composition. When used, suspending agents may comprise 0.01 to 25% by weight of the total composition.

Alcohols, for example, are optional, but may be useful to speed up drying after application to the skin. Alcohol may also be useful as part of a preservative system. Amounts of alcohol up to 5% may be useful. This amount will not interfere will not interfere with the activity of the HDI/trimethylol hexyllactone crosspolymer/silica.

The cosmetic compositions may also comprise ingredients for their preservative and preservative boosting activity, as needed. The total preservative system will typically range from 0.00001% to about 2% by weight of the composition.

Also, pH adjusters, such as sodium hydroxide, may be used as needed to create a consumer acceptable product, typically in the range of 0.00001% to 1% by weight of the composition.

Glycols, also known as diols (chemical compounds comprising two hydroxyl groups) are optional, but sometimes useful in the present invention. Glycols, such as 1,3-propanediol, might typically be used in cosmetics to enhance the freeze-thaw stability of the composition. However, when present glycols may also affect the certain minimum temperature below which the dried composition cannot be easily removed from the skin or hair. Where acrylates copolymer tends to increase the certain minimum temperature, glycols tend to decrease it. Therefore, when used, glycols may be included at 0.00001% to 4% by weight of the total composition, preferably, less than 1% by weight of the total compositions, more preferably less than 0.5% by weight of the total composition. Most preferred is 0% glycols, especially when acceptable freeze-thaw stability is achievable without glycols.

Preferred compositions of the invention have no ingredients that act as surfactants or emulsifiers. However, some surfactant or emulsifier may be useful in some instances to stabilize a hydrophobic powder (i.e. HDI/trimethylol hexyllactone crosspolymer/silica powder) when the powder is being added to an aqueous system, as is the case in the present invention. Therefore, when used, the total of surfactants and emulsifiers should be limited to 0.00001% to 3% by weight of the final composition, and less than would be required to create an emulsion, so that final composition is single phase.

Polyurethane tends to make compositions very rigid, and will alter the certain minimum temperature of water required for removal of the film from the skin or hair. Therefore, it is preferred if compositions of the invention comprise no (0%) polyurethane. If polyurethane is used, its concentration should be limited to 0.001% to 0.5% by weight of the final composition.

Agents that significantly interfere with the structure of the dried film will alter the certain minimum temperature of water required for removal of the film from the skin, as well degrade the color, if present. Therefore, when included, compositions of the invention should be limited to 0.00001% to 0.5% of structuring agents, such as wax, clay (such as bentonite) or stearic acid. More preferably, compositions of the invention are limited to 0.00001% to 0.001% of structuring agents. Most preferably, compositions of the invention comprise no structuring agents (0%). A useful exception to this rule is sodium stearate. Unlike many structuring agents, sodium stearate is partly hydrophilic, which makes it suitable for an aqueous system. Although sodium stearate is partly hydrophobic, its use has not appeared to compromise the objectives of the present invention. Sodium stearate may be used as a structuring agent from 0.00001% to 4% by weight of the total composition.

Any other cosmetic ingredient recognized as safe and that does not interfere with the functional and performance aspects of the compositions of the invention, as herein described, may be included.

The following non-limiting examples illustrate preferred embodiments of the invention.

### Example 1

| | | |
|---|---|---|
| Phase | Ingredient | % Concentration |

| | | | | | |
|---|---|---|---|---|---|
| 1 | water | q.s. | q.s. | q.s. | q.s. |
| 1 | moisturizers | 0.60 | 0.60 | 0.60 | 0.60 |
| 1 | preservative system | 1.85 | 1.85 | 1.85 | 1.85 |
| 2 | thickeners/ | 22.00 | 10.00 | 8.50 | 10.00 |
| | suspending agent(s) | | | | |
| 2 | sodium hydroxide | 0.05 | 0.05 | 0.05 | 0.05 |
| 3 | propanediol | 4.00 | 3.00 | 3.00 | 3.00 |
| 4 | lecithin | 0.05 | 0.05 | 0.05 | 0.05 |
| 4 | HDI/trimethylol | 5.00 | 3.00 | 6.00 | 4.80 |
| | hexyllactone | | | | |
| | crosspolymer//silica | | | | |
| 5 | acrylates/VA | 9.50 | 14.00 | 14.00 | 14.00 |
| | copolymer | | | | |
| 5 | acrylates copolymer | 0.40 | 0.75 | 0.75 | 0.75 |

A preferred procedure for preparing a lotion, serum, cream or sprayable product of the invention is as follows.
1. In a main vessel, sequentially mix phases 1, 2 and 3 until uniform.
2. Separately mix HDI/trimethylol hexyllactone crosspolymer/silica powder with lecithin (or any liquid oil-in-water emulsifier) to coat the powder.
3. Add the step 2 ingredients to the main vessel with mixing.
4. Separately mix and dissolve the acrylates/VA copolymer and acrylates copolymer in a portion of water (this step may be omitted when working with these materials already supplied in solution)
5. Add the step 4 ingredients to the main vessel, mixing well to achieve a uniform mass.

Compositions of the invention are transfer-resistant, water based lotions, serums and creams that provide a mattifying and/or blurring effect on the skin, and vibrant color intensity when pigments are included. Acrylates/VA copolymer and acrylates copolymer form cosmetic compositions that will not crack or flake easily, while not being too stiff for commercial acceptance. At the same time, HDI/trimethylol hexyllactone crosspolymer/silica powder provides the blurring or soft focus effect, which can help to hide fine lines, and provide an overall youthful appearance. We believe that as the composition dries to a clear film, the oil soluble HDI/trimethylol hexyllactone crosspolymer/silica powder becomes trapped in the pores of the film, and are able to scatter light efficiently, which creates the blurring or soft focus effect. Being trapped in the pores of the film, the powder cannot clog pores of the skin.

The compositions provide a mattifying and/or blurring effect, while performing well in terms of smudging and flaking. The compositions have very good break strength and flexibility, are hydrophilic when wet, but hydrophobic when dry. Once applied, the compositions dry in about one minute or less, and can be removed easily with water above a certain minimum temperature, such as 43° C. for example, and scrubbing, but not so easily with water below that temperature, which reduces unwanted transfer and smudging. Even scrubbing with water just one degree below the certain minimum temperature is ineffective to remove thoroughly dried product from skin.

What is claimed is:

1. A single phase, transfer-resistant, aqueous composition comprising, by total weight of the composition:
8% to 16% of acrylates/VA copolymer;
0.2% to 0.8% of acrylates copolymer;
1% to 15% HDI/trimethylol hexyllactone crosspolymer/silica 40% to 70% of water.

2. The composition of claim 1 wherein the ratio of acrylates/VA copolymer to acrylates copolymer is 10:1 to 80:1.

3. The composition of claim 1 wherein the ratio of acrylates/VA copolymer to acrylates copolymer is 12:1 to 40:1.

4. The composition of claim 1 wherein the concentration of HDI/trimethylol hexyllactone crosspolymer/silica is 4% to 8%.

5. The composition of claim 1 further comprising from 0.00001% to 20% of pigments.

6. The composition of claim 1 further comprising 0.00001% to 3% of surfactants and/or emulsifiers.

7. The composition of claim 1 further comprising 0.001% to 0.5% of polyurethane.

8. The composition of claim 1 further comprising 0.00001% to 4% by weight of glycol.
